(19) United States
(12) Patent Application Publication
(10) Pub. No.: US 2025/0332379 A1
(43) Pub. Date: Oct. 30, 2025

(54) CATHETER FOR NAVIGATING TORTUOUS VASCULATURES

(71) Applicant: MicroVention, Inc., Aliso Viejo, CA (US)

(72) Inventors: Jason Su, Aliso Viejo, CA (US); Joseph Gulachenski, Aliso Viejo, CA (US); Steve Tseng, Aliso Viejo, CA (US); Son Pham, Aliso Viejo, CA (US); Raymon Yao, Aliso Viejo, CA (US); Tadele Wolde-Meskel, Aliso Viejo, CA (US)

(73) Assignee: MicroVention, Inc., Aliso Viejo, CA (US)

(21) Appl. No.: 18/994,013

(22) PCT Filed: Jul. 14, 2023
(86) PCT No.: PCT/US2023/070247

§ 371 Date: Jan. 13, 2025

Related U.S. Application Data
(60) Provisional application No. 63/368,463, filed on Jul. 14, 2022.

(51) Int. Cl. A61M 25/00

(52) U.S. Cl. CPC A61M 25/0023; A61M 25/0053; A61M 25/0054; A61M 2025/0063; A61M 2205/0266; A61M 2210/12

(57) ABSTRACT

A catheter for navigating tortuous vasculatures to reach distant anatomies. The catheter may have an optimal stiffness profile so that a physician may safely and efficiently advance large bore catheters through such tortuous vasculatures. The catheter may have a greater stiffness at its proximal end than at its distal end such that the stiffness of the intravascular catheter decreases between its proximal and distal ends. A core wire may extend through proximal and medial segments catheter to impart the desired stiffness. The core wire may be tapered at certain segments and may terminate prior to the distal end of the catheter such that the catheter is more flexible at or near its distal end. The distal end of the core wire may coil around the liner tube of the intravascular catheter. Multiple core wires each having different lengths may be utilized.

### RELATED APPLICATIONS

This application claims benefit of and priority to U.S. Provisional Application Ser. No. 63/368,463 filed Jul. 14, 2022 entitled Catheter For Navigating Tortuous Vasculatures; which is hereby incorporated herein by reference in its entirety.

### BACKGROUND OF THE INVENTION

Treatment of various conditions often requires that a catheter navigate through tortuous vasculatures to reach distant anatomies. For example, treatment of strokes or other conditions may require that a catheter reach distant locations of the patient's vasculature. Reaching such distant vasculatures may often require that the catheter navigate through tortuous vasculatures and will often require that the catheter make hard turns (e.g., turns of 60-180 degrees).

### SUMMARY OF THE INVENTION

An intravascular catheter is described having an optimal stiffness profile for navigating tortuous vasculatures.

One embodiment of the present invention has a decreasing stiffness between its distal and proximal ends.

One embodiment of the present invention has a proximal segment exhibiting a uniform stiffness, a medial segment exhibiting a variable, declining stiffness, and a distal segment exhibiting a uniform stiffness.

One embodiment of the present invention has a higher stiffness in its proximal segment than in its medial segment, and a higher stiffness in its medial segment than in its distal segment.

One embodiment of the present invention includes an internal core wire. The core wire may be integrated along the shaft of the catheter. The material and dimensional profile of the core wire can be manipulated to provide the desired stiffness profile along the catheter shaft. The use of one or more core wires may reduce the need for different durometer polymer jackets or variable pitch braid.

The core wire may function as a guide wire built into the jacket of the catheter. An example embodiment may include such an internal core wire secured along at least a portion of the length of a coiled liner tube. In certain embodiments, the internal core wire may be tapered for at least a portion of its length towards its distal end.

The core wire may terminate at a point which is distally spaced with respect to the distal end of the catheter. As an example, the core wire may terminate in the medial segment of the elongated member of the catheter; with the distal segment of the elongated member remaining more flexible due to the absence of the core wire. The distal segment may comprise approximately 10-12% of the length of the catheter.

In another embodiment of the present invention, the core wire may not terminate into a tapered point so as to avoid potential penetration or piercing of the outer jacket of the catheter. In such embodiments, the core wire may instead be coiled around the liner tube at a distal end of the core wire. In such embodiments, the core wire may have a uniform outer diameter, or may be tapered along at least a portion of its length.

In another embodiment of the present invention, multiple (e.g., two or more) core wires may be positioned and secured against the radial circumference of the liner tube. By way of example, each of four core wires may be secured along the radial circumference of the liner tube at 90-degree increments, with each of the four core wires having different lengths so as to impart a desired, decreasing stiffness profile along the length of the catheter between its proximal and distal ends.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is an elevation view of an intravascular catheter according to one embodiment of the present invention.

FIG. 2 is a partial elevation view of an intravascular catheter employing a tapered core wire according to one embodiment of the present invention.

FIG. 3 is a side view of an intravascular catheter employing a tapered core wire according to one embodiment of the present invention.

FIG. 4 is a cross-sectional view taken along line 4-4 of FIG. 3.

FIG. 5 is a cross-sectional view taken along line 5-5 of FIG. 3.

FIG. 6 is a partial elevation view of an intravascular catheter employing a core wire having a coiled end according to one embodiment of the present invention.

FIG. 7 is a side view of an intravascular catheter employing a core wire having a coiled end according to one embodiment of the present invention.

FIG. 8 is a longitudinal cross-sectional view bisecting a length of a portion of an intravascular catheter according to one embodiment of the present invention.

FIG. 9 is a cross-sectional view taken along line 9-9 of FIG. 7.

FIG. 10 is a partial elevation view of an intravascular catheter employing multiple core wires according to one embodiment of the present invention.

FIG. 11 is a side view of an intravascular catheter employing multiple core wires according to one embodiment of the present invention.

FIG. 12 is a longitudinal cross-sectional view bisecting a length of a portion of an intravascular catheter according to one embodiment of the present invention.

FIG. 13 is a cross-sectional view taken along line 13-13 of FIG. 11.

FIG. 14A is a view illustrating an intravascular catheter traversing a type II aortic arch according to one embodiment of the present invention.

FIG. 14B is a view illustrating an intravascular catheter traversing a type III aortic arch according to one embodiment of the present invention.′

FIG. 15A is a graph comparing stiffnesses along a first range of distances from a distal end of example embodiments of the present invention.

FIG. 15B is a graph comparing stiffnesses along a second range of distances from a distal end of example embodiments of the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The terms distal and proximal are used within this specification. Unless defined otherwise, distal and proximal are used in reference to the physician during a procedure. Hence, proximal tends to be closer to the physician while distal tends to be closer to a target location within a patient. However, this terminology is applicable whether the device is inside or outside of a patient.

An example embodiment of an intravascular catheter 100 may have a decreasing stiffness profile between the proximal and distal ends 104, 105 so as to improve navigation through tortuous vasculatures. The intravascular catheter 100 may include a tubular elongated member 102 having a proximal segment 102A, a medial segment 102B, and a distal segment 102C. A liner tube 107 may extend through the elongated member 102 so as to define a passage 103 (i.e., a lumen) within the elongated member 102.

The proximal segment 102A may have a first stiffness, the medial segment 102B may have a second stiffness, and the distal segment 102C may have a third stiffness. The first stiffness may be greater than the second stiffness, and the second stiffness may be greater than the third stiffness. The first and third stiffnesses may be uniform across their respective segments 102A, 102C, while the second stiffness may decrease (or increase) across at least a portion of the medial segment 102B.

In one example embodiment, the stiffness profile may be imparted to the elongated member 102 through use of a core wire 110 fixed within and extending through an outer jacket 106 of the elongated member 102. The outer diameter of the core wire 110 may be tapered along its length so as to decrease stiffness of the elongated member 102 between its proximal and distal ends 104, 105. The core wire 110 may terminate at or prior to the distal segment 102C of the elongated member 102 such that the distal segment 102C is more flexible than the proximal and medial segments 102A, 102B.

In an example embodiment, a distal end 112 of the core wire 110 may at least partially coil around an outer circumference of the liner tube 107 so as to reduce or eliminate the likelihood of the distal end 112 of the core wire 110 piercing or penetrating the elongated member 102 (e.g., the outer jacket 106 of the elongated member 102). The coiled portion 114 of the core wire 110 may be positioned at or prior to the distal segment 102C of the elongated member 102. In embodiments in which the distal segment 102C of the elongated member 102 includes an enlarged portion 105A, the coiled portion 114 of the core wire 110 may be positioned prior to the proximal end of the enlarged portion 105A.

In another example embodiment, multiple core wires 115A, 115B, 115C, 115D may each be fixed within and extend through the outer jacket 106 of the elongated member 102. The respective lengths of the core wires 115A, 115B, 115C, 115D may be staggered such that each core wire 115A, 115B, 115C, 115D has a different length. In this manner, the stiffness of the elongated member 102 may be gradually decreased along its length. The core wires 115A, 115B, 115C, 115D may be radially positioned about the outer circumference of the liner tube 107. Each core wire 115A, 115B, 115C, 115D may in some embodiments be comprised of a first material 116 and a second material 117 coupled to the first material 116; the first material 116 being stiffer than the second material 117.

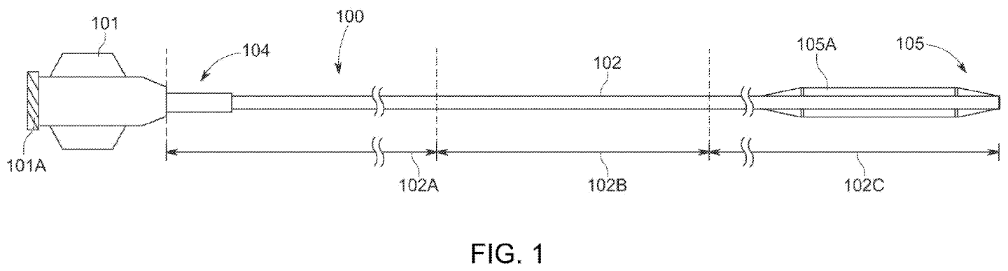

FIG. 1 illustrates one example of an intravascular catheter 100. The methods and systems described herein may be utilized with a wide range of types of catheters 100 and should not be construed as limited to any particular type or configuration of catheter. The catheter 100 may comprise a microcatheter. By way of example, the catheters shown and described in U.S. Pat. No. 10,682,493, titled “Intravascular Treatment Site Access”, and U.S. Pat. No. 10,456,552, titled “System and Methods for Intracranial Vessel Access”, may be utilized. Both U.S. Pat. Nos. 10,682,493 and 10,456,552 are hereby incorporated by reference in their entireties.

The example embodiment of an intravascular catheter 100 illustrated in FIG. 1 may include a hub 101 and an elongated member 102 connected to and extending from the hub 101. The hub 101 may include a passageway 101A that may be fluidly connected to the passage 103 of the catheter 100 such that various tools or devices may be inserted through the passageway 101A and into the passage 103. The passageway 101A of the hub 101 may thus be concentric with the passage 103 of the elongated member 102.

The elongated member 102 may include a proximal end 104 connected to the hub 101 and a distal end 105 which is opposite with respect to the proximal end 104. The elongated member 102 may include an enlarged portion 105A at or near its distal end 105. The enlarged portion 105A may comprise a larger diameter than the remainder of the elongated member 102. The enlarged portion 105A may be positioned exclusively within the distal segment 102C, and thus may not extend into the proximal or medial segments 102A, 102B.

The elongated member 102 may include an outer jacket 106 comprised of various flexible materials including various polymeric materials. Non-limiting examples of such polymeric materials used to form the outer jacket 106 may include thermoplastics such as PEBAX, PET, polytetrafluorethylene (PTFE), polyimide, composites, and the like. The thickness and density of the outer jacket 106 may vary in different embodiments to suit different applications but should be accounted for when designing for an optimal stiffness profile as the outer jacket 106 contributes to the overall stiffness of the catheter 100.

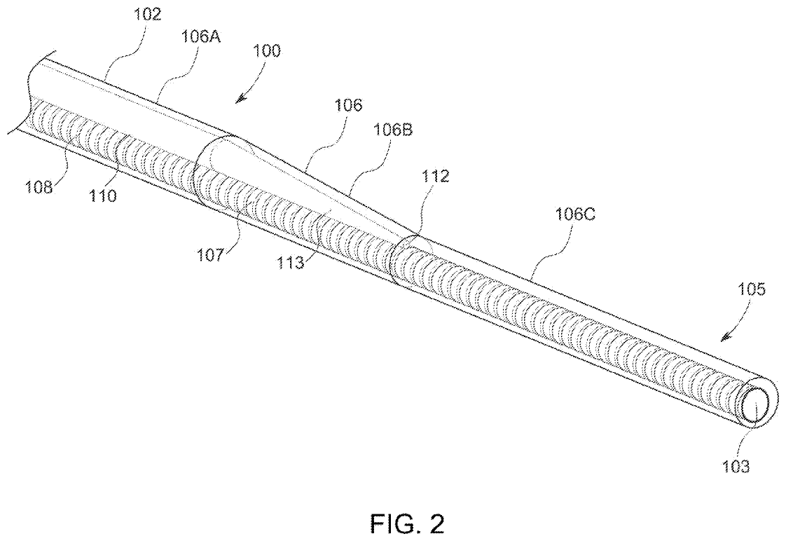

The outer jacket 106 of the elongated member 102 may include different outer diameters along the length of the elongated member 102. In the example embodiments shown in FIGS. 2-3 and 5-7, for example, it can be seen that the outer jacket 106 of the elongated member 102 may include a first segment 106A having a first outer diameter, a second segment 106B having a second outer diameter, and a third segment 106C having a third outer diameter.

The first outer diameter may be greater than the second outer diameter, and the second outer diameter may be greater than the third outer diameter. In the illustrated embodiment, the first segment 106A of the outer jacket 106 may have a uniform first outer diameter, the second segment 106B of the outer jacket 106 may have a tapering (i.e., decreasing) second outer diameter, and the third segment 106C of the outer jacket 106 may have a uniform third outer diameter.

Continuing to reference FIGS. 2-3 and 5-7, it can be seen that the elongated member 102 may only be tapered from a larger diameter to a smaller diameter along a first radial edge. For example, the top of the elongated member 102 may be tapered, with the bottom of the elongated member 102 extending linearly without any taper.

A liner tube 107 may extend through the elongated member 102 so as to define the passage 103, the liner tube 107 having a diameter which may be less than that of the elongated member 102 through which it extends. The passage 103 may extend through the liner tube 107. In this manner, the elongated member 102 may define an outer tube and the liner tube 107 may define an inner tube extending through the outer tube.

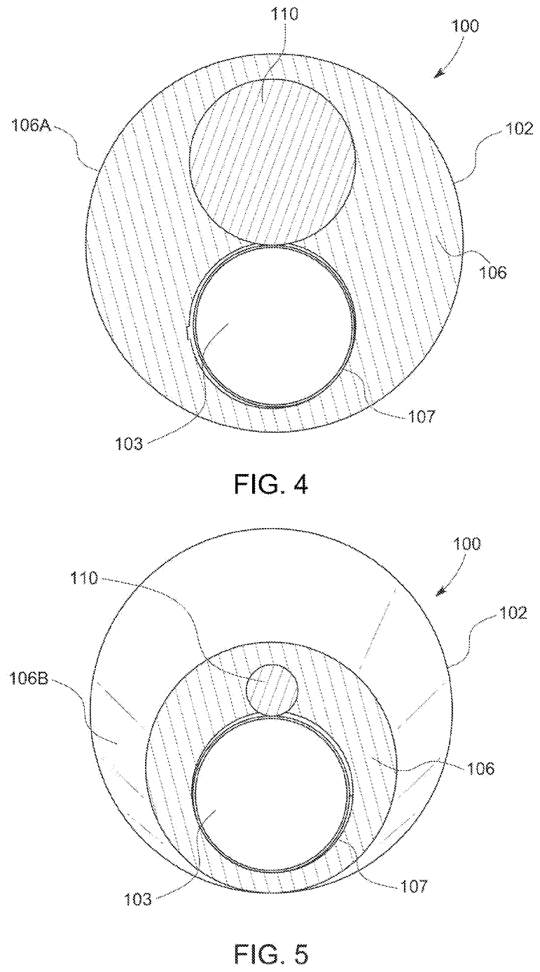

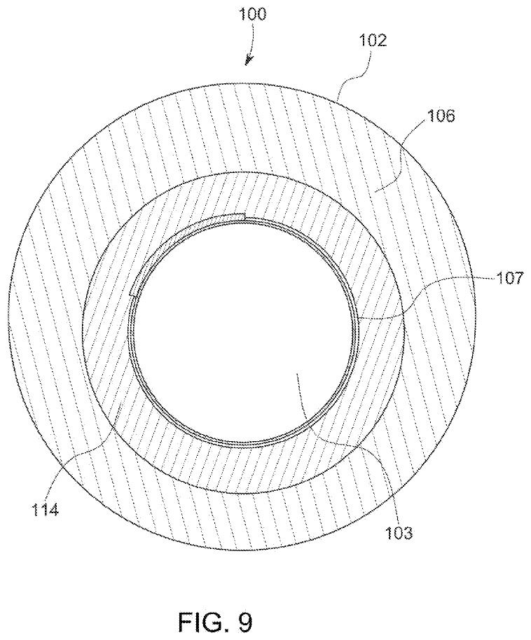

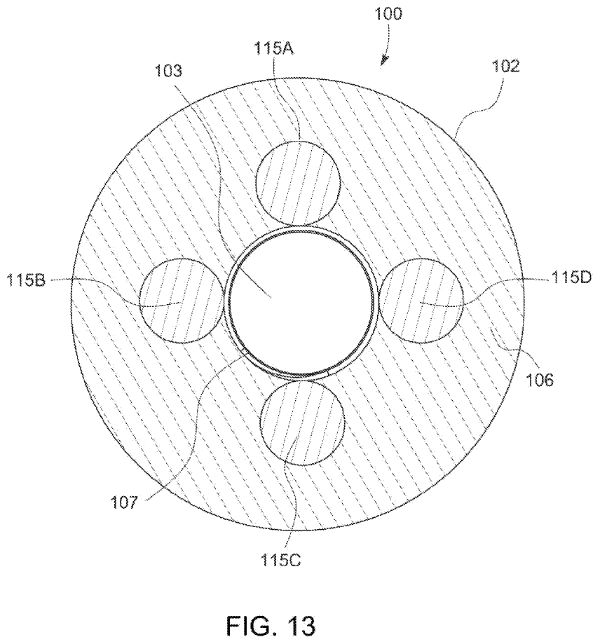

The positioning of the passage 103 within the elongated member 102 may vary in different embodiments. For example, FIGS. 4 and 5 illustrate an example embodiment in which the liner tube 107 may extend through a lower half of the height of the outer jacket 106 of the elongated member 102. Put differently, the passage 103 may be positioned below a longitudinal axis extending through a center of the elongated member 102. FIGS. 9 and 13 illustrate an example embodiment in which the passage 103 may be centrally positioned within the elongated member 102.

The respective diameters of the elongated member 102 and passage 103, and the ratio between them, may vary in different embodiments to suit different applications. Thus, the respective sizes of the elongated member 102 and passage 103, and the ratio therebetween, should not be construed as limited by the exemplary embodiments illustrated in the figures.

The length of the liner tube 107, and thus the passage 103, may be equal to that of the elongated member 102, though in some embodiments the liner tube 107 may be of greater or lesser length than the elongated member 102. The liner tube 107 may be fixed within the elongated member 102 such that the liner tube 107 is not removable therefrom. The liner tube 107 may be coiled as shown in the figures, with a coiled wire 108 being coiled around at least a portion of the length of the liner tube 107. The pitch of the coiled wire 108 may vary in different embodiments. The pitch of the coiled wire 108 may be uniform across the length of the liner tube 107 or may vary along different portions of the length of the liner tube 107.

As illustrated in FIG. 1, the elongated member 102 of the catheter 100 may include a proximal segment 102A, a medial segment 102B, and a distal segment 102C. The proximal segment 102A may comprise a first length of the elongated member 102 between its proximal end 104 and the medial segment 102B. The medial segment 102B may comprise a second length of the elongated member 102 positioned between the proximal and distal segments 102A, 102C. The distal segment 102C may comprise a third length of the elongated member 102 between the medial segment 102B and the distal end 105 of the elongated member 102.

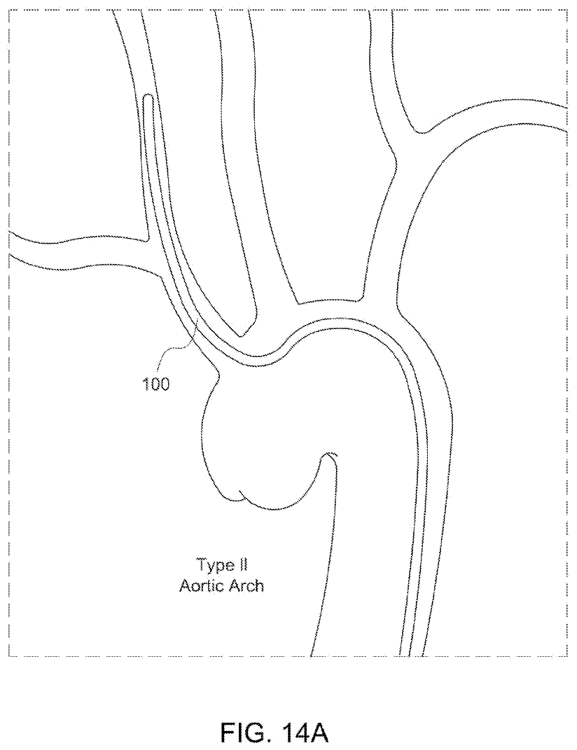

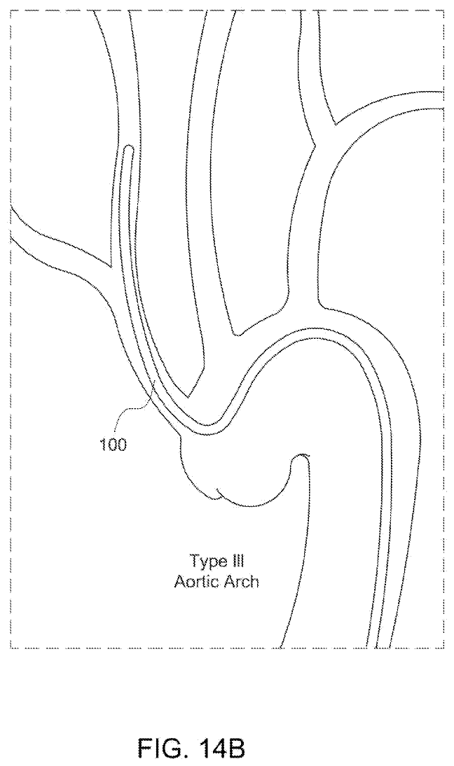

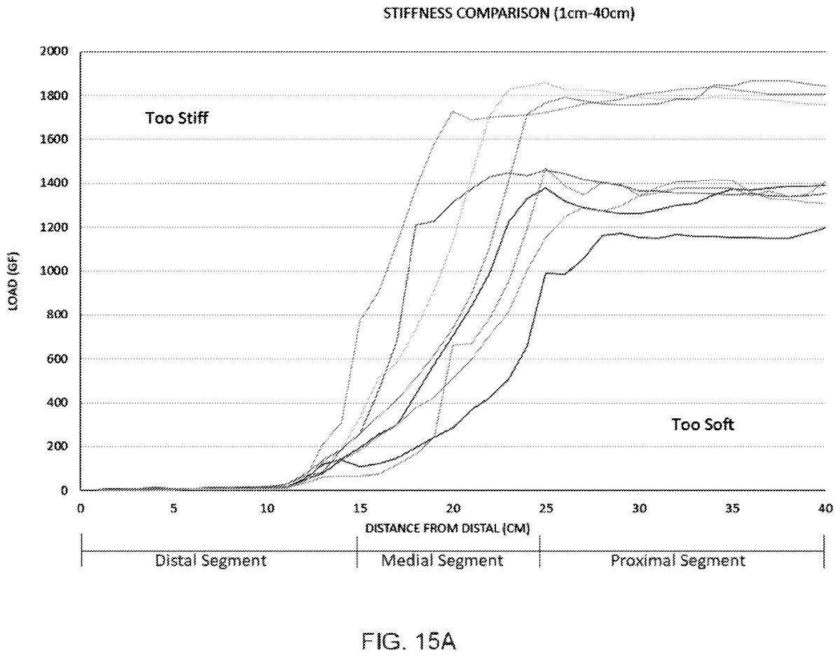

In an example embodiment as shown in FIG. 1, the distal end 105 of the elongated member 102 may include an enlarged portion 105A having a greater diameter than the remainder (e.g., the proximal and medial segments 102A, 102B) of the elongated member 102, though in some embodiments the enlarged portion 105A may be omitted such as shown in FIGS. 14A and 14B.

The lengths of each of the segments 102A, 102B, 102C may vary in different embodiments. In various illustrated embodiments shown in the figures, the length of the proximal segment 102A may be greater than the length of the medial segment 102B. The length of the distal segment 102C may be greater than the length of the medial segment 102B.

The ratio of the respective lengths of the different segments 102A, 102B, 102C may vary in different embodiments. In an exemplary embodiment, the sum of the lengths of the proximal, medial, and distal segments 102A, 102B, 102C, and thus the overall length of the elongated member 102, may be between 150-165 centimeters. However, such dimensions are merely for exemplary purposes, as the elongated member 102 could be longer or shorter depending on the application for which the catheter 100 is used.

In one example embodiment, the length of the medial segment 102B may be between 6-16 centimeters as measured between the proximal and distal segments 102A, 102C. The length of the distal segment 102C may be between 12-26 centimeters as measured from the distal end 105 of the elongated member 102 towards the proximal end 104. Thus, in one example embodiment, the length of the distal segment 102C may be between approximately 7%-16% of the length of the elongated member 102 of the catheter 100.

In an example embodiment, the length of the elongated member 102 may be between 150 centimeters and 165 centimeters, and the length of the distal segment 102C, into which the core wire 110 may not extend as discussed below, may be between 15 centimeters and 20 centimeters as measured from a distal end 105 of the elongated member 102 towards a proximal end 104 of the elongated member 102. In another example embodiment, the combined length of the proximal, medial, and distal segments 102A, 102B, 102C may be 165 centimeters, and the length of the distal segment 102C may be 16.5 centimeters.

Such dimensions, in which a specific length of the distal segment 102C is left more flexible than the proximal and medial segments 102A, 102B, have been found to provide an optimal stiffness profile for navigating tortuous vasculatures. For example, the increased flexibility (and thus decreased stiffness) of the final 7%-16% of the length of the elongated member 102 ensures that the distal segment 102C may be easily manipulated to traverse hard turns within a vasculature. By omitting the core wire 110 from the final 7%-16% of the length of the elongated member 102, the distal end 105 of the elongated member 102 may be left more flexible for hooking around or otherwise navigating through tortuous vasculatures having hard turns.

Each of the preceding lengths are merely for illustrative purposes and should not be construed as limiting in scope, as different lengths (and ratios between lengths) may be utilized in different embodiments to suit different applications and procedures.

The elongated member 102 of the catheter 100 may be a uniform diameter across its length or, in some example embodiments such as shown in FIGS. 2-3 and 6-7, may be tapered along one or more segments 102A, 102B, 102C of its length. Thus, the uniform diameter of the elongated member 102 shown in, e.g., FIGS. 1, 14A, and 14B, should not be construed as limiting in scope.

In at least the example embodiments shown in FIGS. 2-3 and 5-7, it can be seen that the elongated member 102 of the catheter 100 may be tapered along at least a portion of the length of the medial segment 102B. In such an embodiment, the outer jacket 106 of the elongated member 102 may include successive first, second, and third segments 106A, 106B, 106C each having different outer diameters; with the first segment 106A having a uniform first outer diameter, the second segment 106B having a tapered second outer diameter which may be less than the first outer diameter, and the third segment 106C having a uniform third outer diameter which may be less than the first and second outer diameters.

In some embodiments, the taper of the diameter of the elongated member 102 may continue along at least a portion of the length of the distal segment 102C. In some embodiments, the medial segment 102B of the elongated member 102 may be tapered and the proximal and distal segments 102A, 102B of the elongated member 102 may be a uniform diameter. In further example embodiments, the proximal segment 102A of the elongated member 102 may be a uniform diameter and both the medial and distal segments 102B, 102C may be tapered. In further example embodiments, the distal segment 102C may include an enlarged portion 105A as discussed herein, with the enlarged portion 105A having a greater diameter than any other portion of the elongated member 102.

The intravascular catheter 100 will generally have an optimal stiffness profile for traversing tortuous vasculatures such as Type Il or IlI aortic arches. The intravascular catheter 100 may have a variable stiffness profile between its proximal and distal ends 104, 105. Each of the respective segments 102A, 102B, 102C may have different stiffnesses. The distal segment 102C may have less stiffness (i.e., be more flexible) than the proximal and medial segments 102A, 102B such that the distal segment 102C may be easily hooked, curved, or otherwise manipulated to make hard turns through tortuous vasculatures.

The stiffness of the intravascular catheter 100 may decrease (e.g., ramp down) between the proximal and distal ends 104, 105 of the elongated member 102 in an example embodiment. In such an example embodiment, the rate of decrease in stiffness of the intravascular catheter 100 may be uniform across its length. In another example embodiment, the rate of decrease in stiffness of the intravascular catheter 100 may be variable, e.g., by having both portions with uniform stiffness and portions with decreasing stiffness.

In an example embodiment, the intravascular catheter 100 may have a first stiffness along the length of the proximal segment 102A. The first stiffness of the elongated member 102 along the length of the proximal segment 102A may be uniform (i.e., consistent for the length of the proximal segment 102A). However, in other embodiments, the first stiffness may increase or decrease along the proximal segment 102A of the intravascular catheter 100. The proximal segment 102A may comprise a proximal length of the elongated member 102 extending from the proximal end 104 and terminating at the medial segment 102B.

In an example embodiment, the intravascular catheter 100 may have a second stiffness along the length of the medial segment 102B. The second stiffness may be less than the first stiffness. Thus, the stiffness of the medial segment 102B may be less than the stiffness of the proximal segment 102A such that the stiffness of the elongated member 102 decreases between the proximal and medial segments 102A, 102B. The second stiffness may be uniform across the medial segment 102B, or the stiffness may decrease (or increase) at various rates along the length of the medial segment 102B. The medial segment 102B may comprise a medial length of the elongated member 102 between the proximal and distal ends 104, 105. The medial segment 102B may comprise a length of the elongated member 102 extending from the proximal segment 102A to the distal segment 102C (i.e., the medial segment 102B extends between the proximal and distal segments 102A, 102C of the elongated member 102).

In an example embodiment, the intravascular catheter 100 may have a third stiffness along the length of the distal segment 102C. The third stiffness may be less than the second stiffness. Thus, the stiffness of the distal segment 102C may be less than the stiffness of each of the proximal and medial segments 102B, 102C. The third stiffness of the intravascular catheter 100 may be uniform across the distal segment 102C (i.e., consistent for the length of the distal segment 102C), or the stiffness may decrease (or increase) at various rates along the length of the distal segment 102C. The distal segment 102C may comprise a distal length of the elongated member 102 extending from the medial segment 102B and terminating at the distal end 105.

The manner by which the desired stiffness profile of the intravascular catheter 100 is achieved may vary in different embodiments. In a first example embodiment, a core wire 110 may be connected to the intravascular catheter 100 in a fixed manner to impart the desired stiffness profile to the catheter 100. Thus, the core wire 110 may extend through the intravascular catheter 100 in a non-removable manner. For example, the core wire 110 may be integrated into the outer jacket 106 of the elongated member 102. The core wire 110 may extend through a lumen in the elongated member 102 that is separate and distinct from the passage 103 of the elongated member 102. The lumen of the elongated member 102 through which the core wire 110 extends may be parallel to the passage 103 of the elongated member 102. The core wire 110 may in some embodiments be connected to the liner tube 107 either at one or more points along the length of the core wire 110 or consistently along its length.

The stiffness of the intravascular catheter 100 may be the sum of the stiffnesses of the outer jacket 106, the liner tube 107, and the core wire 110. The core wire 110 may thus be configured in different embodiments to achieve a desired stiffness for traversing tortuous vasculatures. In some embodiments, the core wire 110 may terminate at the distal end of the medial segment 102B (i.e., the core wire 110 terminates at the start of the distal segment 102C). In such embodiments, the stiffness of the distal segment 102C may be the sum of the stiffness of the outer jacket 106 and coiled liner tube 107 since the core wire 110 is absent in that segment 102C.

In the example embodiments shown in the figures, the desired stiffness profile of the intravascular catheter 100 may be achieved through use of a core wire 110 extending at least partially through the intravascular catheter 100. In an example embodiment, the core wire 110 may be tapered along at least one of the segments 102A, 102B, 102C of the elongated member 102. In another example embodiment, the core wire 110 may transition into a coiled portion 114 such that a distal end 112 of the core wire 110 coils around the passage 103. In other embodiments, multiple core wires 115A, 115B, 115C, 115D of varying lengths and/or materials 116, 117 may extend through the intravascular catheter 100; the multiple core wires 115A, 115B, 115C, 115D being positioned outside of the liner tube 107.

In embodiments in which the core wire 110 is tapered along at least a portion of its length, the length and degree of taper may vary in different embodiments. In one example embodiment, the core wire 110 is a uniform outer diameter along the proximal segment 102A and tapered along the medial segment 102B. In one example embodiment, the outer diameter of the distal end 112 of the core wire 110 at the medial segment 102B (e.g., after its taper) may be between 12%-14% of the starting outer diameter of the core wire 110 at the proximal segment 102A.

By way of example, the outer diameter of the core wire 110 along the proximal segment 102A may be between 0.05 and 0.07 centimeters. The outer diameter of the core wire 110 may then taper along the medial segment 102B down to an outer diameter of between 0.007 and 0.008 centimeters. The length of the taper of the outer diameter of the core wire 110 may be between 7-16 centimeters. Such configurations have been found to be optimally suited for traversing hard turns in tortuous vasculatures.

Each of the preceding dimensions are merely for illustrative purposes and should not be construed as limiting in scope, as different lengths and degrees of tapers may be utilized in different embodiments of the core wire 110 to suit different applications.

The material(s) 116, 117 of the core wire 110 may vary in different embodiments. In one example embodiment, the core wire 110 may be stainless steel. In another example embodiment, the core wire 110 may be a nickel-titanium alloy (i.e., nitinol). In yet another example embodiment, the core wire 110 may comprise both stainless steel and nitinol.

In some embodiments, the core wire 110 may comprise both stainless steel and nitinol. For example, the core wire 110 along the proximal segment 102A may be comprised of stainless steel and the core wire 110 along the medial segment 102B may be comprised of nitinol. Such an arrangement allows for more stiffness towards the proximal end 104 and more flexibility towards the distal end 105.

The core wire 110 will generally be integrated or seated within the outer jacket 106 of the elongated member 102. Thus, the core wire 110 may be fixed within the elongated member 102 such that the core wire 110 is not removable. The core wire 110 may be secured or positioned against the liner tube 107 (e.g., against the outer circumference of the passage 103) as shown in the figures, though in some embodiments the core wire 110 may instead extend parallel to the liner tube 107 without contacting the liner tube 107.

In embodiments in which the core wire 110 may be secured against the liner tube 107, the manner by which the core wire 110 may be secured against the liner tube 107 may vary. While adhesives such as glue may be utilized to secure the core wire 110 against the liner tube 107, the adhesive will generally affect the stiffness profile of the elongated member 102 and thus must be accounted for in such embodiments. In one example embodiment, a heat shrink tube may be used to secure the core wire 110 against the liner tube 107, and the liner tube 107 within the outer jacket 106. In such a preferred embodiment, a mandrel or the like may be inserted within the liner tube 107 to maintain the passage 103 when the heat shrink tube is applied.

FIGS. 2-5 illustrate an example embodiment of an intravascular catheter 100 having a decreasing stiffness profile between its proximal and distal ends 104, 105. In the illustrated embodiment, an elongated core wire 110 may extend through the intravascular catheter 100. The core wire 110 may be secured against or in contact with the passage 103 (e.g., the outer circumference of the liner tube 107). In an example embodiment, the core wire 110 may be secured against the upper end of the outer circumference of the liner tube 107. However, in other embodiments, the core wire 110 may be secured at various other radial positions about the outer circumference of the liner tube 107 (e.g., the bottom or sides). In other embodiments, the core wire 110 may not be secured against or in contact with the liner tube 107, but instead may extend parallel to the liner tube 107 within the outer jacket 106.

The core wire 110 may be the same length as the elongated member 102, or it may be longer or shorter than the elongated member 102. In one example embodiment, a proximal end of the core wire 110 may extend out of the proximal end 104 of the elongated member 102 and into the passageway 101A of the hub 101. In an example embodiment, approximately one centimeter of length of the core wire 110 may extend past the proximal end 104 of the elongated member 102 and into the passageway 101A of the hub 101 of the catheter 100. Such a configuration ensures that manipulation of the hub 101 by a physician is also imparted to the core wire 110.

In the example embodiments of the figures, the core wire 110 is illustrated as comprising a uniform outer diameter for its length along the proximal segment 102A of the elongated member 102. The outer diameter of the core wire 110 along the proximal segment 102A of the elongated member 102 may be comprised of various sizes. In an example embodiment, the outer diameter may be between 0.05 and 0.07 centimeters.

The core wire 110 thus may have a first stiffness along the proximal segment 102A of the elongated member 102. The core wire 110 may maintain the same outer diameter, and thus the same stiffness, along an entire length of the proximal segment 102A of the elongated member 102 from the proximal end 104 to the medial segment 102B.

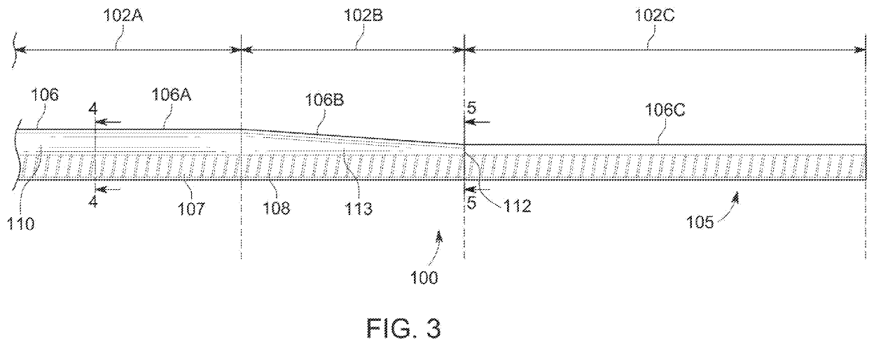

As best shown in FIGS. 2 and 3, the core wire 110 may comprise a tapered portion 113 having a decreasing outer diameter (i.e., a taper) for its length along the medial segment 102B of the elongated member 102. The rate of decrease in outer diameter of the tapered portion 113 of the core wire 110 (e.g., the decrease in outer diameter per length) along the medial segment 102B may vary in different embodiments and should not be construed as limited by the example embodiments shown in the figures. The tapered portion 113 may extend for the entire length of the medial segment 102B such as shown in FIGS. 2-3, or in other embodiments may extend for less than or greater than the length of the medial segment 102B.

In the embodiment shown in FIGS. 2 and 3, the core wire 110 may terminate at its distal end 112 at the transition between the medial and distal segments 102B, 102C of the elongated member 102. Thus, the distal end 112 of the core wire 110 may terminate prior to the start of the enlarged portion 105A of the elongated member 102 such that the enlarged portion 105A has sufficient flexibility to traverse hard turns in tortuous vasculatures.

The distal end 112 of the core wire 110 may have an outer diameter which is less than the outer diameter of the core wire 110 along the proximal segment 102A of the elongated member 102. As an example, the outer diameter of the distal end 112 of the core wire 110 may be between 12%-14% of the outer diameter of the core wire 110 along the proximal segment 102A of the elongated member 102. In an example embodiment, the outer diameter of the distal end 112 of the core wire 110 may be between 0.007 and 0.008 centimeters. Such a configuration ensures that the stiffness of the core wire 110 ramps down along the length of the elongated member 102 to provide an optimal stiffness profile for navigating through tortuous vasculatures including hard turns.

Continuing to reference FIGS. 2 and 3, it can be seen that the core wire 110 may not extend into the distal segment 102C of the elongated member 102. Such a configuration ensures flexibility of the distal segment 102C and distal end 105 of the elongated member 102 so as to allow for the manipulation necessary to navigate tortuous vasculatures such as through an aortic arch. Thus, in such an embodiment, the core wire 110 may not contribute any stiffness to the distal segment 102C of the elongated member 102, with the overall stiffness of the distal segment 102C being the sum of the stiffnesses of the outer jacket 106 and liner tube 107 (including any coiled wire 108 coiled around the liner tube 107).

FIGS. 4 and 5 illustrate cross-sectional views of the elongated member 102 to illustrate both the liner tube 107 and the core wire 110. FIG. 4 is a cross-sectional view taken along line 4-4 of FIG. 3 within the proximal segment 102A of the elongated member 102. As shown in FIG. 4, the outer diameter of the core wire 110 may be approximately the same as the outer diameter of the liner tube 107. However, it should be appreciated that, in other example embodiments, the outer diameter of the core wire 110 may be greater than or less than the outer diameter of the liner tube 107 so as to achieve different desirable stiffness profiles.

With reference to FIG. 4, it can be seen that the passage 103, and thus the liner tube 107, may be positioned below a longitudinal axis extending through a center of the elongated member 102 along at least a portion of the length of the elongated member 102. Similarly, it can be seen that the core wire 110 may be positioned above the longitudinal axis extending through the center of the elongated member 102. Such a configuration may improve navigation through a tortuous vasculature having hard turns.

FIG. 5 is a cross-sectional view taken along line 5-5 of FIG. 3 at the termination of the medial segment 102B of the elongated member 102 and thus at the distal end 112 of the core wire 110 after the taper. As shown in FIG. 5, the outer diameter of the distal end 112 of the core wire 110 may be less than the outer diameter of the liner tube 107. In one example embodiment, the outer diameter of the distal end 112 of the core wire 110 may be approximately 12%-14% of the outer diameter of the liner tube 107. It can also be seen in FIG. 5 that the outer jacket 106 of the elongated member 102 may also be inwardly tapered along a length of its second segment 106B to match the taper of the core wire 110 along the medial segment 102B.

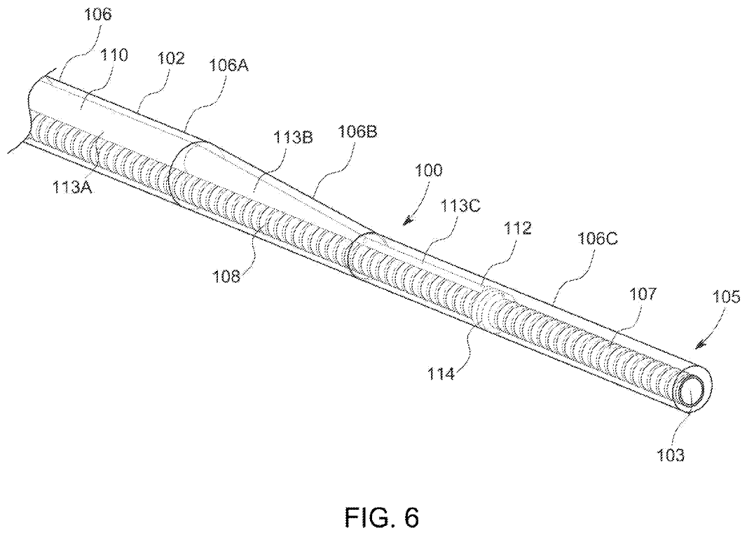

FIGS. 6-9 illustrate an example embodiment of an intravascular catheter 100 having a decreasing stiffness profile between its proximal and distal ends 104, 105. As with the previous embodiment, an elongated core wire 110 may extend through the intravascular catheter 100. The core wire 110 may be secured against the outer circumference of the passage 103, e.g., by being secured against the outer circumference of the liner tube 107. Although the figures illustrate the core wire 110 being secured against or in contact with the upper end of the outer circumference of the liner tube 107, the core wire 110 in other embodiments may be secured at or in contact with various other radial positions about the outer circumference of the liner tube 107. In other embodiments, the core wire 110 may not be in contact with or secured against the liner tube 107 but instead may extend parallel to the liner tube 107 within the outer jacket 106.

As with the previous embodiment, the core wire 110 may comprise a uniform outer diameter for its length along the proximal segment 102A of the elongated member 102. Various outer diameters may be utilized in different embodiments to suit different applications and exhibit different desired stiffness profiles. In an example embodiment, the outer diameter of the core wire 110 along the proximal segment 102A may be between 0.05 and 0.07 centimeters.

The core wire in the embodiment shown in FIGS. 6-9 may have a uniform first stiffness along the proximal segment 102A of the elongated member 102. Thus, the core wire 110 as shown in FIGS. 6-9 may maintain the same outer diameter and the same stiffness along an entire length of the proximal segment 102A of the elongated member 102 from the proximal end 104 to the medial segment 102B.

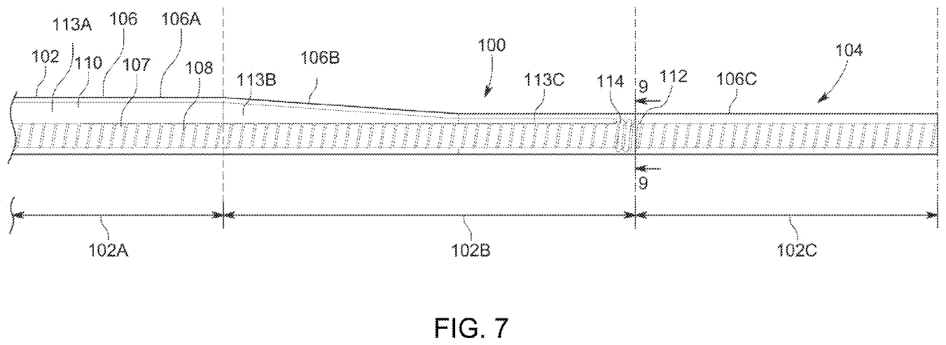

As best shown in FIGS. 6 and 7, at least a portion of the core wire 110 along the length of the medial segment 102B of the elongated member 102 may have a decreasing outer diameter (i.e., a taper). The rate of decrease in outer diameter of the core wire 110 along the medial segment 102B may vary in different embodiments and should not be construed as limited by the example embodiments shown in the figures.

In an example embodiment such as shown in FIGS. 6 and 7, the core wire 110 may include a first segment 113A having a first uniform outer diameter. The first segment 113A of the core wire 110 having the first uniform outer diameter may extend from the proximal end 104 of the elongated member 102 for the length of its proximal segment 102A; terminating at the medial segment 102B of the elongated member 102.

The core wire 110 may include a second segment 113B having a second tapered outer diameter in which the outer diameter of the core wire 110 may decrease. The second segment 113B may extend for a portion of the medial segment 102B. The degree of taper (i.e., the rate and amount of decreasing outer diameter) may vary in different embodiments.

The core wire 110 may include a third segment 113C having a third uniform outer diameter. The third segment 113C may extend for a portion of the medial segment 102B. The third segment 113C may terminate into a coiled portion 114 in which the distal end 112 of the core wire 110 coils around the liner tube 107 such as shown in the figures. The coiled portion 114 may have a helical shape which extends around the outer circumference of the liner tube 107 for a varying number of turns in different embodiments.

The first, second, and third segments 113A, 113B, 113C of the core wire 110 may be integral. The third uniform outer diameter of the third segment 113C may be less than the first uniform outer diameter of the first segment 113A. The second tapered outer diameter of the second segment 113B may taper from the first uniform outer diameter to the third uniform outer diameter.

Thus, it can be seen that as the core wire 110 extends across the medial segment 102B of the elongated member 102, the core wire 110 may include a tapered second segment 113B having a tapered outer diameter and a uniform third segment 113C having a uniform outer diameter that is less than the outer diameter of the first segment 113A of the core wire 110 in the proximal segment 102A of the elongated member 102.

The respective lengths of the tapered second segment 113B and the uniform third segment 113C of the core wire 110 as they extend along the medial segment 102B of the elongated member 102 may vary in different embodiments. In some embodiments, the core wire 110 may taper across the entire length of the medial segment 102B of the elongated member 102 prior to terminating in a coiled portion 114 as discussed below. In such embodiments, the uniform third segment 113C may be omitted. In other embodiments, the core wire 110 may retain a uniform outer diameter across the entire length of the medial segment 102B of the elongated member 102 prior to terminating in a coiled portion 114.

As shown in FIGS. 6-8, the core wire 110 may terminate into a coiled portion 114 in which the distal end 112 of the core wire 110 is coiled around the liner tube 107. Such an embodiment may be desirable to eliminate the risk of the distal end 112 of the core wire 110 penetrating through the outer jacket 106 of the elongated member 102, such as when taking a hard turn, and injuring the patient. By coiling the core wire 110 around the liner tube 107, there is a substantially reduced risk of the distal end 112 of the core wire 110 puncturing or penetrating the elongated member 102.

With reference to FIGS. 6-7, it can be seen that the core wire 110 may not extend into or across any portion of the distal segment 102C of the elongated member 102. Thus, the distal segment 102C of the elongated member 102 may have a greater flexibility (i.e., lesser stiffness) than both the proximal and medial segments 102A, 102B. Such a configuration may aid in making hard turns as are common when navigating tortuous vasculatures such as those found in a typical aortic arch.

In the example embodiments shown in the figures, it can be seen that the coiled portion 114 of the core wire 110 may include approximately three turns around the liner tube 107. However, it should be appreciated that the coiled portion 114 may include more or less turns in different embodiments. Further, it should be appreciated that the pitch of the coiled portion 114 may vary in different embodiments and should not be construed as limited by the example embodiments shown in the figures. Additionally, while the figures illustrate that the coiled portion 114 shares the same outer diameter as the uniform third segment 113C of the coil wire 110, in some embodiments the coiled portion 114 may taper as it coils around the liner tube 107.

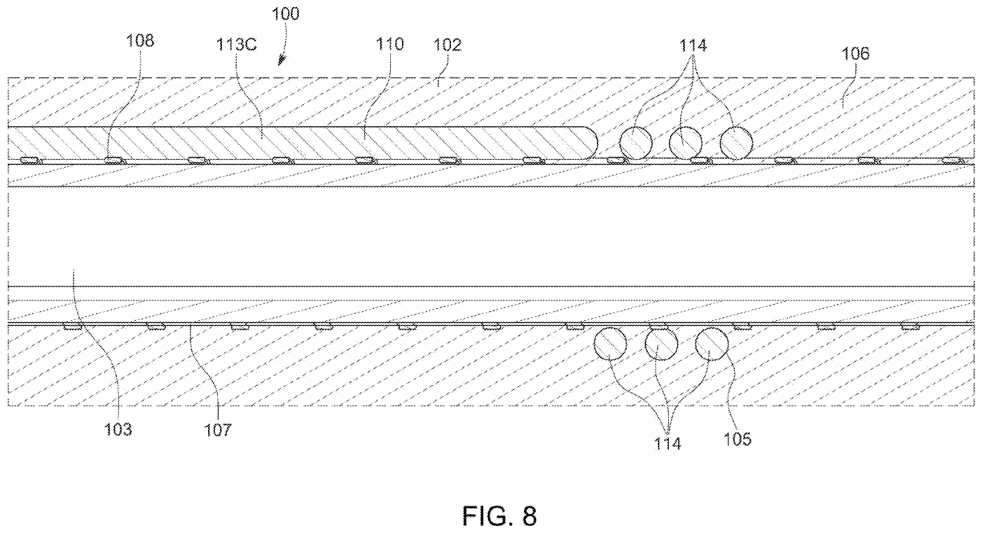

FIG. 8 illustrates a longitudinal cross-sectional view bisecting the length of a portion of the elongated member 102. In the illustrated example embodiment, it can be seen that the core wire 110 may be secured against or in contact with the top of the liner tube 107 prior to terminating with the coiled portion 114 coiling around the liner tube 107. In other embodiments, the core wire 110 may instead extend along other radial positions along the outer circumference of the liner tube 107 than the top.

FIG. 9 is a cross-sectional view taken along line 9-9 of FIG. 7. As shown in FIG. 9, the coiled portion 114 may coil completely around the liner tube 107 so as to terminate the coil wire 110 without coming to a point or leaving an exposed, linear distal end 112 that could potentially pierce or penetrate the outer jacket 106 of the elongated member 102. However, in some embodiments, the coiled portion 114 may coil only partially around the liner tube 107 (e.g., the coiled portion 114 may have less than a full turn). By way of example, the coiled portion 114 may only coil around half of the outer circumference of the liner tube 107, and thus have only half a turn, in some embodiments.

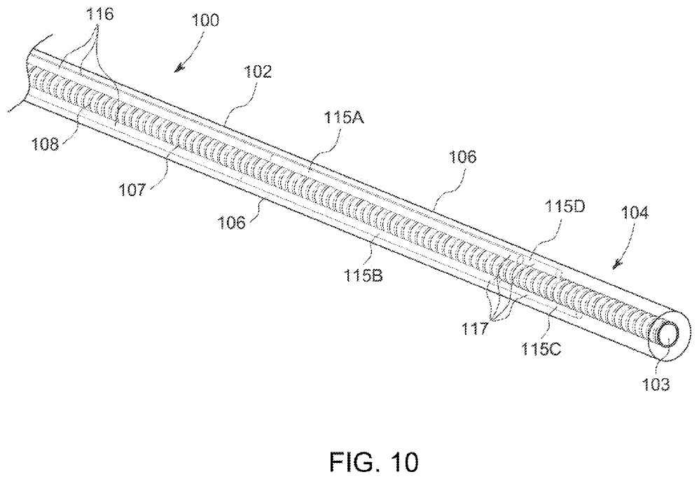

FIGS. 10-13 illustrate a third exemplary embodiment of an intravascular catheter 100 having multiple core wires 115A, 115B, 115C, 115D rather than a single core wire 110. Such an embodiment may utilize core wires 115A, 115B, 115C, 115D having a uniform outer diameter for each of their lengths, rather than any tapering. However, in some example embodiments, one or more of the core wires 115A, 115B, 115C, 115D may taper along at least a portion of their respective lengths.

In the illustrated embodiment, each of four core wires 115A, 115B, 115C, 115D extend through the intravascular catheter 100. It should be appreciated that more or less core wires 115A, 115B, 115C, 115D may be utilized in different embodiments to achieve the desired stiffness profile. For example, in one example embodiment, only two core wires 115A, 115B may be utilized. In other embodiments, three, five, or more core wires 115A, 115B, 115C, 115D may be utilized.

Each of the core wires 115A, 115B, 115C, 115D is illustrated as being secured against or in contact with the outer circumference of the liner tube 107. In the illustrated example embodiment as best shown in FIGS. 10 and 13, a first core wire 115A may be secured against or in contact with the top of the liner tube 107, a second core wire 115B may be secured against or in contact with a first side of the liner tube 107, a third core wire 115C may be secured against or in contact with the bottom of the liner tube 107, and a fourth core wire 115D may be secured against or in contact with the second side of the liner tube 107. In other embodiments, one or more of the core wires 115A, 115B, 115C, 115D may not be secured against or in contact with the liner tube 107, but instead may extend parallel to the liner tube 107 within the outer jacket 106.

In the embodiment shown in the figures, each of the core wires 115A, 115B, 115C, 115D may be positioned radially about the outer circumference of the liner tube 107 at equal distances with respect to each other. More specifically, it can be seen that each of the core wires 115A, 115B, 115C, 115D may be separated by ninety degrees from adjacent core wires 115A, 115B, 115C, 115D. However, in certain embodiments, the spacing of the core wires 115A, 115B, 115C, 115D about the outer circumference of the liner tube 107 may vary. For example, two more of the core wires 115A, 115B, 115C, 115D could be grouped closer together.

As best shown in FIGS. 10-12, each of the core wires 115A, 115B, 115C, 115D may have a different length. The different lengths of the core wires 115A, 115B, 115C, 115D may function to gradually decrease or ramp down the stiffness of the catheter 100 across the medial segment 102B of the elongated member 102. Thus, by terminating each core wire 115A, 115B, 115C, 115D at a different position along the medial segment 102B, the effective stiffness may be decreased gradually prior to the termination of the final core wire 115A, 115B, 115C, 115D at the start of the distal segment 102C.

In the example embodiment shown in FIG. 10, the third core wire 115C may be longer than each of the first, second, and fourth core wires 115A, 115B, 115D, the fourth core wire 115D may be longer than the each of the first and second core wires 115A, 115B, and the first core wire 115A may be longer than the second core wire 115B. However, such a configuration is merely shown as an example embodiment as various other alternate arrangements may be utilized in different embodiments.

While the figures illustrate that each of the core wires 115A, 115B, 115C, 115D are different lengths with respect to each other, two or more of the core wires 115A, 115B, 115C, 115D may have the same length in certain embodiments. Further, the positioning of the longest and shortest core wires 115A, 115B, 115C, 115D shown in the figures is merely for illustrative purposes, and thus should not be construed as limiting in scope. For example, the figures illustrate that the third core wire 115C, which may extend along and be secured to the bottom of the liner tube 107, is the longest of the core wires 115A, 115B, 115C, 115D and that the first core wire 115A, which may extend along and be secured to the top of the liner tube 107, is the shortest of the core wires 115A, 115B, 115C, 115D. However, the inverse configuration, or various other configurations, may be alternatively used in different embodiments.

As a further aid to controlling the stiffness profile of the catheter 100, one or more of the core wires 115A, 115B, 115C, 115D may comprise different materials. The figures illustrate that each of the core wires 115A, 115B, 115C, 115D is comprised of two materials 116, 117. The first material 116, forming a proximal length of each core wire 115A, 115B, 115C, 115D, may be a more rigid or stiff material such as stainless steel. The second material 117, which may be coupled to the distal end of the first material 116 to form a unitary core wire 115A, 115B, 115C, 115D and may form a distal length of each core wire 115A, 115B, 115C, 115D, may be a less rigid (i.e., more flexible) material such as nitinol.

The length of the respective materials 116, 117 used for each core wire 115A, 115B, 115C, 115D may vary in different embodiments. While the figures illustrate that the second material 117 may comprise a shorter length of each core wire 115A, 115B, 115C, 115D than the first material 116, the inverse could be utilized in certain embodiments. In another example embodiment, the lengths of the respective first and second materials 116, 117 may be equal. In some embodiments, the entirety of each core wire 115A, 115B, 115C, 115D may comprise a single material 116, 117. In other embodiments, three or more materials may be fused together to form each core wire 115A, 115B, 115C, 115D.

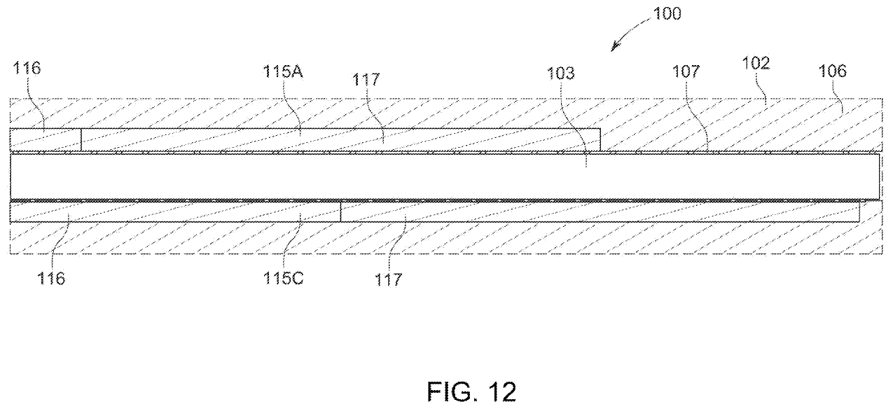

FIG. 12 is a longitudinal cross-sectional view bisecting the length of a portion of the elongated member 102. In the illustrated embodiment, the first core wire 115A is illustrated as being secured against and running along the top of the liner tube 107 and the third core wire 115C is illustrated as being secured against and running along the bottom of the liner tube 107. The respective sizes of the core wires 115A, 115B, 115C, 115D and liner tube 107 may vary from what is shown in the figures in different embodiments.

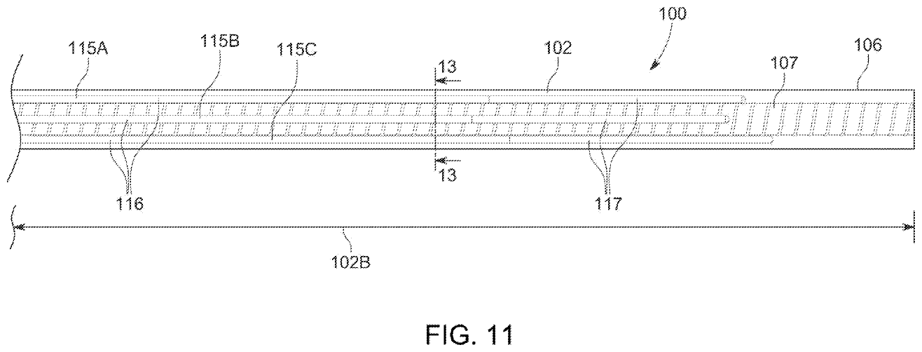

FIG. 13 is a cross-sectional view taken along line 13-13 of FIG. 11. As shown in FIG. 13, each of the four core wires 115A, 115B, 115C, 115D may be radially positioned about the outer circumference of the liner tube 107. The core wires 115A, 115B, 115C, 115D may be secured directly against or in contact with the liner tube 107 or may instead be seated within the outer jacket 106 in a fixed manner parallel to, but not in contact with, the liner tube 107. As this view is for merely an illustrative example, it should be appreciated that the ratio between the diameter of the core wires 115A, 115B, 115C, 115D and that of the liner tube 107 may vary in different embodiments.

FIGS. 14A and 14B illustrate an example intravascular catheter 100 navigating or traversing through an aortic arch. FIG. 14A illustrates a Type II aortic arch and FIG. 14B illustrates a Type III aortic arch. The example intravascular catheter 100 shown in FIGS. 14A and 14B is simplified for illustration purposes, and it should be appreciated that such an intravascular catheter 100 may include an enlarged portion 105A, or segments 106A, 106B, 106C of its outer jacket 106 having different outer diameters, as discussed previously.

In operation, the intravascular catheter 100 may be routed up through the descending aorta and through the arch of aorta. The intravascular catheter 100 may then make a hard right turn to enter the brachiocephalic artery. The catheter 100 may have a more flexible distal segment 102C to allow the distal segment 102C, and thus the distal end 105, of the catheter 100 to traverse through such hard turns more easily. As shown in the illustrated embodiment, the intravascular catheter 100 may continue into the right common carotid artery.

The optimal stiffness profile of the intravascular catheter 100, in which the stiffness of the catheter 100 may decrease along its length between its proximal and distal ends 104, 105 and may include a flexible distal segment 102C, may allow a physician to more easily and efficiently navigate the catheter 100 through the aortic arch with a reduced or eliminated risk of the catheter 100 kicking back into the ascending aorta. While the figures illustrate navigation through the aortic arch and into the right common carotid artery, it should be appreciated that various other routes may be utilized with the systems and methods described herein. For example, the catheter 100 may instead be routed through the right subclavian artery, left common carotid artery, or left subclavian artery; with each of these routes being made possible due to the unique and optimal stiffness profile of the catheter 100.

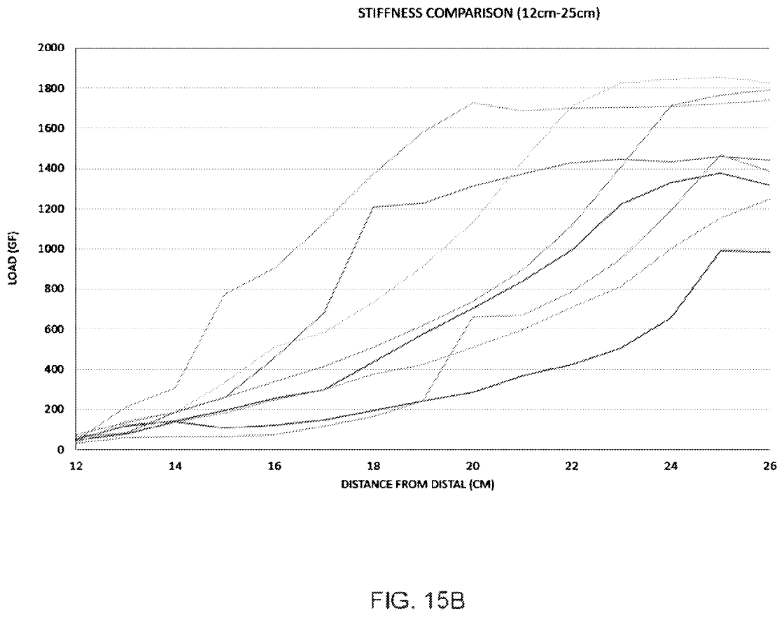

FIGS. 15A and 15B illustrate exemplary graphs comparing stiffnesses along different distances ranges from the distal end 105 of example embodiments of a catheter 100. Both figures compare load values in gram-force (GF) to distances from the distal end 105 of example embodiments of catheters 100 in centimeters (cm). FIG. 15A illustrates load values in GF for a range between 0 cm and 40 cm from the distal end 105 of example embodiments of a catheter 100. FIG. 15B illustrates load values in GF for a range between 12 cm and 26 cm from the distal end 105 of example embodiments of a catheter 100.

As shown in FIG. 15A, example embodiments of a catheter 100 may have a negligible load values (e.g, less than 50 GF) between 0-10 cm from the distal end 105. Load values tend to increase between approximately 12 cm from the distal end 105 to approximately 25 cm from the distal end 105, and then flatten out between approximately 25 cm from the distal end 105 to approximately 40 cm from the distal end 105. The rate and degree of load increase between the 25 cm and 40 cm distances vary in different illustrated embodiments, with each illustrated embodiment having different rates, degrees, and lengths of taper along the distal segment 102C of the catheter 100.

FIG. 15B focuses on the critical range between 25 cm and 40 cm from the distal end 105 of the various example embodiments shown in FIG. 15A. As shown in FIG. 15B, depending on characteristics including the rate, degree, and length of taper along the distal segment 102C of the catheter 100, the load will generally increase between less than 100 GF to between just below 1000 GF and just greater than 1800 GF.

As previously discussed, the proximal segment 102A of an example embodiment of a catheter 100 may have a first stiffness, the medial segment 102B may have a second stiffness less than or equal to the first stiffness, and the distal segment 102C may have a third stiffness less than or equal to the first stiffness; with the second stiffness decreasing along a length of the medial segment 102B from the first stiffness to the second stiffness.

As shown in FIGS. 15A-15B, the first stiffness may be greater than 1000 GF (e.g., between 1000 GF and 1850 GF) and the third stiffness may be less than 100 GF (e.g., between 0 GF and 100 GF such as, in some embodiments, less than 50 GF). A length of the distal segment 102C may be less than 20 cm as measured from the distal end 105 of the catheter 100 towards its proximal end 104. A length of the medial segment 102B may be less than 25 cm as measured from the proximal end of the distal segment 102C of the catheter 100 to a distal end of the proximal segment 102A of the catheter 100. However, it should be appreciated that such values are merely for exemplary and illustrative purposes, and thus should not be construed as limiting in scope.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An intravascular catheter, comprising:
an elongated member having a proximal segment, a medial segment, and a distal segment, the distal segment of the elongated member having an enlarged portion with a diameter greater than the proximal and medial segments;
a passage extending through the elongated member; and
a first core wire within the elongated member adjacent to the passage, a distal end of the first core wire terminating prior to the enlarged portion of the elongated member.

2. The intravascular catheter of claim 1, wherein a length of the distal segment of the elongated member is between 7%-16% of a length of the elongated member, and wherein the first core wire does not extend into the distal segment.

3. (canceled) The intravascular catheter of claim 2, wherein a length of the elongated member is between 150 centimeters and 165 centimeters, and wherein the length of the distal segment is between 15 centimeters and 20 centimeters as measured from a distal end of the elongated member towards a proximal end of the elongated member.

4. The intravascular catheter of claim 1, the first core wire being secured against an outer circumference of the passage.

5. (canceled)

6. (canceled)

7. The intravascular catheter of claim 1, wherein a proximal end of the first core wire extends out of a proximal end of the elongated member.

8. The intravascular catheter of claim 1, wherein a distal end of the first core wire is at least partially coiled around the passage.

9. The intravascular catheter of claim 1, wherein the first core wire has a linear segment and a coiled segment, the linear segment extending parallel to the passage and the coiled segment being coiled around the passage.

10. The intravascular catheter of claim 9, wherein the coiled segment of the first core wire is positioned within the medial segment of the elongated member.

11. (canceled)

12. The intravascular catheter of claim 1, further comprising a second core wire within the elongated member adjacent to the passage, wherein the first core wire is a different length than the second core wire.

13. The intravascular catheter of claim 12, wherein a distal end of the second core wire terminates prior to the enlarged portion of the elongated member.

14. The intravascular catheter of claim 12, further comprising a third core wire within the elongated member adjacent to the passage and a fourth core wire within the elongated member adjacent to the passage.

15. The intravascular catheter of claim 14, wherein the first core wire, the second core wire, the third core wire, and the fourth core wire are each positioned radially about the passage.

16. The intravascular catheter of claim 14, wherein the first core wire, the second core wire, the third core wire, and the fourth core wire each have different lengths.

17. The intravascular catheter of claim 14, wherein the first core wire, the second core wire, the third core wire, and the fourth core wire each have a first segment comprised of stainless steel and a second segment comprised of nitinol.

18. The intravascular catheter of claim 1, wherein the proximal segment has a first stiffness, the medial segment has a second stiffness less than or equal to the first stiffness, and the distal segment has a third stiffness less than or equal to the first stiffness, and
wherein the second stiffness decreases along a length of the medial segment from the first stiffness to the third stiffness.

19. The intravascular catheter of claim 18, where the first stiffness is at least 1,000 grams-force (GF) and the third stiffness is less than 50 grams-force (GF).

20. The intravascular catheter of claim 19, wherein a length of the distal segment is less than 20 centimeters as measured from a distal end of the elongated member towards a proximal end of the elongated member.

21. The intravascular catheter of claim 20, wherein the length of the medial segment is less than 25 centimeters as measured from a proximal end of the distal segment to a distal end of the proximal segment.

22. An intravascular catheter, comprising:
an elongated member having a proximal segment, a medial segment, and a distal segment, the distal segment of the elongated member having an enlarged portion with a diameter greater than the proximal and medial segments;
a passage extending through the elongated member; and
a stiffening means within the elongated member adjacent to the passage, wherein a distal end of the stiffening means terminates prior to the enlarged portion of the elongated member.

23. The intravascular catheter of claim 22, wherein the stiffening means is comprised of a core wire, wherein the core wire includes a linear segment extending parallel to the passage and a coiled segment coiled around the passage.

24. The intravascular catheter of claim 22, wherein the stiffening means is comprised of a plurality of core wires, wherein each of the plurality of core wires has a different length.
